(19) United States
(12) Patent Application Publication
(10) Pub. No.: US 2013/0039929 A1
(43) Pub. Date: Feb. 14, 2013

(54) METHOD TREATING BREAST CANCER

(71) Applicants: Sudha Shenoy, Durham, NC (US); Mark Dewhirst, Durham, NC (US); Sang-Oh Han, Durham, NC (US)

(72) Inventors: Sudha Shenoy, Durham, NC (US); Mark Dewhirst, Durham, NC (US); Sang-Oh Han, Durham, NC (US)

(21) Appl. No.: 13/642,324

(22) PCT Filed: Apr. 19, 2011
(86) PCT No.: PCT/US11/00693

§ 371 Date: Oct. 19, 2012

Related U.S. Application Data
(60) Provisional application No. 61/282,904, filed on Apr. 19, 2010.

(51) Int. Cl. A61K 31/713; C07D 401/04; C07H 21/02; A61K 39/395; A61P 35/00

(52) U.S. Cl. 424/172.1; 514/44 A; 536/24.5; 546/200

(57) ABSTRACT

The present invention relates, in general, to breast cancer and, in particular, to methods of treating breast cancer comprising administering to a subject in need thereof an agent that modulates signal transduction regulated by β-arrestin (e.g., β-arrestin 1). The invention further relates to methods of identifying compounds suitable for use in such methods.

This application claims priority from U.S. Provisional Appln. No. 61/282,904, filed Apr. 19, 2010.

This invention was made with government support under Grant No. HL080525 awarded by the National Institutes of Health. The government has certain rights in the invention.

### TECHNICAL FIELD

The present invention relates, in general, to breast cancer and, in particular, to methods of treating breast cancer comprising administering to a subject in need thereof an agent that modulates signal transduction regulated by β-arrestin (e.g., β-arrestin 1). The invention further relates to methods of identifying compounds suitable for use in such methods.

### BACKGROUND

G-protein-coupled receptors (GPCRs), also known as 7 transmembrane-spanning receptors (7TMRs), are a family of cell surface proteins capable of binding a myriad of extracellular ligands and initiating various signaling cascades within the cell (for review see DeWire et al, Annu. Rev. Physiol. 69:483-510 (2007)). Due to their relative abundance, GPCRs now account for nearly 50% of currently marketed drugs (Ma et al, Nat. Rev. Drug Discov. 1:571-572 (2002)). The traditional paradigm of GPCR signaling involves the transduction of extracellular signals through the binding of ligand to the extracellular surface of the receptor. This binding is thought to induce a conformational change in the cytoplasmic surface of the receptor which allows for the activation of heterotrimeric G-protein complexes and generation of second messengers such as cyclic AMP and diacylglycerol kinase.

Activation of G-proteins also recruits a class of kinases, known as the G-protein coupled receptor kinases (GRKs), to the receptor to initiate the termination of G-protein-dependent signaling. GRKs rapidly phosphorylate the receptor, and this phosphorylation triggers the recruitment and binding of the unique molecular scaffold, β-arrestin.

There are four members of the arrestin family. Visual arrestin, or arrestin 1, is localized to retinal rods, whereas X arrestin, or arrestin 4, is found in retinal rods and cones. β-arrestin1 (aka arresting) and β-arrestin2 (aka arrestin3) are ubiquitously expressed multifunctional signaling adaptor proteins originally discovered for their role in desensitizing GPCRs (Lefkowitz and Shenoy, Science 308:512-517 (2005)). β-arrestins regulate both GPCR and non-GPCR pathways, under normal as well as pathological conditions including cancer (Lefkowitz et al, Mol. Cell 24:643-652 (2006)).

The two β-arrestin isoforms share roughly 70% sequence identity and, in general, perform similar functions in GPCR regulation (for example, receptor desensitization) (Moore et al, Annu. Rev. Physiol. 69:451-482 (2007), Kohout et al, Proc. Natl. Acad. Sci. USA 98:1601-1606 (2001)). However, recent studies utilizing siRNA-mediated depletion and individual isoform repletion of the β-arrestin1/2 null mouse embryonic fibroblasts have revealed differential roles in the extent of their endocytic and signaling functions with respect to some GPCRs (Kohout et al, Proc. Natl. Acad. Sci. USA 98:1601-1606 (2001), Aim et al, Proc. Natl. Acad. Sci. USA 100:1740-1744 (2003)). Reports also indicate that the two isoforms can function in a reciprocal manner to regulate GPCR signaling (DeWire et al, Annu. Rev. Physiol. 69:483-510 (2007)). Of the two β-arrestin isoforms, β-arrestin2 is excluded from the nucleus due to the presence of an NES or Nuclear Export Signal, that is absent in β-arrestin1 (Scott et al, J. Biol. Chem. 277:37693-37701 (2002), Wang et al, J. Biol. Chem. 278:11648-11653 (2003), Kang et al, Cell 123:833-847 (2005)).

### SUMMARY OF THE INVENTION

The present invention relates generally to breast cancer. More specifically, the invention relates to methods of treating breast cancer comprising administering to a subject in need thereof an agent that modulates signal transduction regulated by β-arrestin. The invention further relates to methods of identifying compounds suitable for use in such methods.

Objects and advantages of the present invention will be clear from the description that follows.

### BRIEF DESCRIPTION OF THE DRAWINGS

FIGS. 1A-1E. FIG. 1A. Indicated amounts of cell extracts were analyzed by Western blotting using the rabbit polyclonal antibodies anti-β-arrestin1 (A1CT, top panel) and anti-β-arrestin2 (A2CT, middle panel) generated against carboxyl terminal domains of β-arrestin1 and β-arrestin2, respectively. The two antibodies have five-fold more affinity toward the cognate antigen isoform than the other. The bottom panel shows relative amounts of ERK 1 and 2 (as a loading control) in the same lysate samples. FIG. 1B. Indicated amounts of cell extracts of MDAMB-231 and normal breast epithelial cells (Hs 578Bst, ATCC) were immunoblotted for β-arrestins with A1CT antibody (top panel) and ERK (lower panel). FIG. 1C. Protein bands corresponding to the 10 μg input were quantified from three to four independent experiments, normalized to protein (μg) input and plotted as bar graphs. ** p<0.01, carcinoma cells versus others, one-way ANOVA, Bonferroni post test. FIG. 1D. Immunostaining of human breast tissue sections (Zymed breast tissue arrays) for βarr1 expression. Representative confocal micrographs shown were obtained using LSM 510 microscope; identical instrumental settings were used to acquire images for both samples. FIG. 1E. The pixel intensity for Garr immunostaining from sections for normal and invasive ductal carcinoma (IDC) was quantified using MetaMorph, normalized to DNA and plotted as bar-graph. ** p=0.0084, t test, two-tailed, n=10.

FIGS. 2A-2D. Breast carcinoma cells with stable luciferase expression (231-luc) were transfected with control or βarr1/2 targeting siRNA and then injected into nude mice 50 h later. The spread of luciferase-tagged cells was determined by in vivo bioluminescence imaging after D-luciferin i.p. injection. The time course of luminescence representing tumor growth is shown in FIG. 2A. One representative mouse each from ‘control-cells’ group and ‘βarrestin-depleted’ group are shown for the indicated time points. FIG. 2B. Quantification of luminescence using the Living Image acquisition and analysis software (Xenogen). * p<0.05 by Two-way ANOVA. FIG. 2C. Western blot analyses of lysates of respective samples of injected cells. The top bands are nonspecific bands, which also serve as loading controls. FIG. 2D. Luciferase activity of respective aliquots of control and ‘βarr1/2’ cells that were used for injections, as assayed with a luminometer.

FIGS. 3A and 3B. FIG. 3A. MDAMB-231 cells transfected with siRNA targeting no mRNA, βarr1 or βarr2 were plated on 96-well opaque plate without or with 100 μM CoCl₂. 20,000 cells were plated in a volume of 100 μl. 24 hours later equal volume of CellTiter-Glo® reagent (Promega) was added, plates were shaken for 5 min and luminescence was measured with a plate reader for 0.5 sec/well. Cell viability was calculated as percentage ATP present according to the manufacturer's protocol. The data presented are mean±SEM from three experiments. * p<0.05 and ** p<0.01 versus control-hypoxia, one-way ANOVA, Bonferroni post test. FIG. 3B. Western blot analyses showing the efficiency of siRNA-mediated knockdown of individual isoforms.

FIGS. 4A-4D. FIG. 4A. Endogenous βarr from untreated or CoCl₂-treated breast carcinoma cells (MDAMB-231) was immunoprecipitated with an anti-βarr antibody and the immunoprecipitates (IPs) were probed with anti-HIF-1α antibody. Representative blots are shown from one of two similar experiments. FIG. 4B. Confocal images depict immunostaining for β-arrestin1 (green) and HIF-1α (red) in MDAMB-231 cells treated with CoCl₂. FIG. 4C. MDAMB-231 cells were transfected with indicated plasmids encoding Flag-tagged β-arrestins. The top panel shows the amount of HIF-1α bound to Flag-βarr IPs. The middle panel shows the amount of βarr in each IP sample. Lowest panel displays detection of HIF-1α in CoCl₂-treated lysate samples. FIG. 4D. HIF-1α in βarr IP was quantified and normalized to βarr levels. ***p<0.001, versus βarr1, Bonferroni post test, one-way ANOVA, n-=4.

FIGS. 5A-5E. FIG. 5A. Schematic map of luciferase reporter used; HRE: hypoxia responsive element. FIG. 5B. Assay of hypoxia-induced luciferase activity in the presence of each indicated siRNA transfection. **p<0.001, * p<0.01 versus control/CoCl₂ one-way ANOVA, Bonferroni post test. FIG. 5C. Western blot showing the efficiency of knockdown for each βarr isoform. FIG. 5D. Assay of hypoxia induced luciferase reporter activity in Mouse Embryonic Fibroblasts (MEFs) that are null for both βarr1 and 2 under control and βarr1 replete conditions. *** p<0.001, between the two cobalt treated samples, Bonferroni post test, one-way ANOVA, n=3. FIG. 5E. Western blot of lysates showing expression of transfected βarr1.

FIGS. 6A-6B. FIG. 6A. Confocal micrographs showing β-arrestin1 (green), VEGF-A (red) and DNA labeled with DRAQ5™ (blue) from normal breast tissue (top panels), infiltrating ductal carcinoma, IDC, (middle panels) and metastatic-IDC, from lymph nodes (lowest panels). FIG. 6B. Signals from each channel were quantified using MetaMorph and plotted as bar graphs. Both β-anestin1 and VEGF-A levels were increased more than 3-fold and significantly higher (* p<0.05, ** p<0.01) in IDC n=40 tissue samples, 80 images) than in normal breast tissues (n=10 tissue samples, 22 images).

FIGS. 7A-7D. FIG. 7A. MDAMB-231 cells were transfected with 5×-HRE-luciferase and after indicated treatment, the extent of transcriptional activity was determined as in FIG. 5. FIG. 7B. Cells were treated as indicated and whole cell extracts were analyzed for HIF-1α by Western blotting. FIG. 7C. Untreated or thalidomide (10 μM) treated MDAMB-231 cells were immunostained for β-arrestin levels and confocal images were obtained as in FIG. 4B. FIG. 7D. MDAMB-231 cells were treated for 5 hours with CoCl₂ alone or CoCl₂ plus thalidomide, fixed, immunostained for βarrestin (A1CT) and HIF-1α and analyzed by confocal microscopy.

### DETAILED DESCRIPTION OF THE INVENTION

β-arrestin1 gene maps to chromosome locus 11q13, which is amplified in breast cancer and the protein is up-regulated in breast carcinoma cells as well as in infiltrating ductal carcinoma (IDC). Depletion of β-arrestin1 in invasive breast carcinoma retards tumor colonization in nude mice and prevents cellular growth in vitro under hypoxic conditions. (See Example that follows.) β-arrestin1 and not β-arrestin2 robustly interacts with the hypoxia-inducible factor-1α (HIF-1α) subunit stabilized during hypoxia. This interaction is crucial for HIF-1 dependent transcription measured by a 5×-HRE (hypoxia response elements) luciferase reporter. Furthermore, increase in β-arrestin1 expression in IDC and metastatic IDC correlates with increased levels of VEGFA, an angiogenic transcriptional target of HIF-1. While the immunomodulatory and antiangiogenic drug thalidomide inhibits HIF-1 dependent transcription in breast carcinoma cells, it does not prevent HIF-1α stabilization. However, thalidomide induces cytoplasmic transport of β-arrestin1, as well as aberrant localization of HIF-1α to the perinuclear compartments of breast carcinoma cells. These findings indicate that β-arrestin1 is an important regulator of signaling during hypoxia and that drugs that induce its translocation from the nucleus to the cytoplasm can be useful in the treatment of breast cancer. (See Example below.)

The present invention relates generally to methods of treating breast cancer comprising administering to a subject in need thereof an agent that modulates β-arrestin-dependent signaling. In one aspect, the invention relates to methods of treating breast cancer comprising administering agents that inhibit signal transduction regulated by β-arrestin (e.g., β-arrestin1). In another aspect, the invention relates to methods of identifying inhibitors suitable for use in such methods.

Inhibitors of the invention include any pharmaceutically acceptable agent that can bind β-arrestin (e.g., β-arrestin1) and modify (e.g., inhibit/disrupt) the interaction between β-arrestin and its signaling partners, or which can degrade, metabolize, cleave or otherwise chemically alter β-arrestin so that signal transduction is inhibited or disrupted. Inhibitors of the invention also include agents that can inhibit expression of β-arrestin.

Examples of inhibitors of the invention include small molecules, oligonucleotides (e.g., aptamers, siRNAs, miRNAs, or aptamer/siRNA chimeras), and proteins (e.g., antibodies or binding fragments thereof (e.g., Fab fragments)). Aptamers capable of binding to β-arrestin (e.g., β-arrestin1) in a manner such that interaction of β-arrestin with its signaling partners is inhibited/disrupted can be produced using techniques known in the art (see, for example, Tuerk and Gold, Science 249:505-510 (1990), Ellington and Szostak, Nature 346:818-822 (1990), Guo et al, Int. J. Mol. Sci. 9(4):668-768 (2008), Lee and Sullenger, Nat. Biotechnol. 15(10:41-45 (1997), Que-Gewirth and Sullenger, Gene Ther. 14(4):283-291 (2007), Nimjee et al, Trends Cardiovasc. Med. 15(1):41-45 (2005), U.S. Pat. No. 5,270,163). SiRNAs or miRNAs appropriate for use in inhibiting expression of β-arrestin (e.g., β-arrestin1) can also be designed and produced using protocols known in the art (Elbashir et al, Nature 411:494-498 (2001), Fire et al, Nature 391:806-811 (1998), Hammond et al, Nature 404:293-295 (2000), Han et al, Cell 125(5):887-901 (2006), see also US Published Appln. No. 20040053411). Monoclonal antibodies (e.g., humanized or chimeric) specific for β-arrestin (e.g., β-arrestin1), as well as binding fragments thereof (e.g., Fab fragments), can be prepared using protocols well known in the art (Winter et al, Annul. Rev. Immunol. 12:433-455 (1994), Fellouse et al, J. Mol. Biol. 373(4):924-940 (2007), Epub 2007 Aug. 19; Sidhu et al, Curr. Opin. Struct. Biol. 17(4):481-487 (2007), Epub 2007 Sep. 17; Jia et al, Int. J. Biol. Sci. 4(2):103-10 (2008)). Of particular interest in connection with the present invention are synthetic antibody fragments purified from phage display libraries and selected according to their affinity to bind to β-arrestin (e.g., β-arrestin1) (Fellouse et al, J. Mol. Biol. 373:924-940 (2007), Ye et al, Proc. Natl. Acad. Sci. USA 105:82-87 (2008), Sidhu et al, Curr. Opin. Struct. Biol. 17:481-487 (2007), Rizk et al, Proc. Natl. Acad. Sci. USA 106:11011-11015 (2009)).

Small molecule inhibitors suitable for use in the invention can be identified by screening candidate compounds in an assay that measures binding of the compound to β-arrestin1 (and/or 2). Alternatively, assays (in vitro or in vivo) that measure the difference in β-arrestin-dependent signaling in the presence and absence of the candidate small molecule can be used.

Methods have been developed to monitor conformational changes that occur in β-arrestins in response to ligand binding. These methods include fluorescence resonance energy transfer (FRET)-based assays and bioluminescent resonance energy transfer (BRET)-based assays (see, for example, Shukla et al, Proc. Natl. Acad. Sci. 105:9988-9993 (2008) and Charest et al, EMBO reports 6(4):334340 (2005)). Such assays can be used to monitor conformational changes that occur upon binding of candidate compounds in the binding assays described above. Once a small molecule is identified that binds to β-arrestin in a manner that induces a conformational change associated with inhibition of β-arrestin signaling or the prevention of complex formation between β-arrestin and its binding partners, techniques (such as combinatorial approaches) can be used to optimize the chemical structure for the desired inhibitory effect.

Crystal structures are known for certain β-arrestins (Han et al, Structures 9(9):869-80 (2001); Milano et al, Biochemistry 41(10):3321-8 (2002); Sutton et al, J. Mol. Biol. 354(5):1069-80 (2005), Epub 2005 Nov. 2; Granzin et al, Nature 391(6670):918-21 (1998)). Accordingly, structure-based design strategies can be used to produce small molecule inhibitors of β-arrestin. Such inhibitors can target, for example, an arrestin fold or an arrestin domain which are shared among the family members. (See, for example, Gurevich et al, Structure 11(9):1037-42 (2003), Aubry et al, Curr. Genomics 10(2):133-142 (2009), Gurevich et al, Genome Biol. 7(9):1236 (2006).)

The inhibitors of the invention can be targeted to appropriate sites in vivo either by appropriate selection of the route of administration or by the use of targeting moieties (Khandare et al, Crit. Rev. Ther. Drug Carrier Syst. 23(5):401-35 (2006), Martin et al, AAPS J. 9(1):E18-29 (2007)). For example, aptamers specific for molecules over-expressed on the surface of target cells can be used to deliver inhibitors of the invention (including oligonucleotide inhibitors). Also, delivery methods have been developed that are suitable for use in connection with the present invention for the transport of proteins to the cytoplasm of mammalian cells without disrupting the integrity of the cell membrane (Rizik et al, Proc. Natl. Acad. Sci. USA 106:11011-11015 (2009); Michiue et al, J. Biol. Chem. 280(9):8285-9 (2005), Epub 2004 Dec. 16; Sugita et al, Biochem. Biophys. Res. Commun. 363(4):1027-32 (2007), Epub 2007 Sep. 29; Gump et al, J. Biol. Chem. 285(2):1500-7 (2010, Epub 2009 Oct. 26)).

The invention further relates to compositions comprising inhibitors of the invention formulated with an appropriate carrier. The composition can be in dosage unit form (e.g., a tablet or capsule suitable, for example, for oral administration). The composition can also be present, for example, as a solution or suspension (e.g., a sterile solution or suspension) suitable, for example, for injection. Further, the composition can take the form of a gel, cream or ointment, e.g., suitable for topical administration.

The optimum amount or any particular inhibitor to be administered can be readily determined by one skilled in the art. That amount can vary with the inhibitor, the patient (human or non-human mammal) and the effect sought.

Certain aspects of the invention can be described in greater detail in the non-limiting Example that follows.

### Example

### β-Arrestin1 is Up-Regulated in Invasive Breast Carcinoma

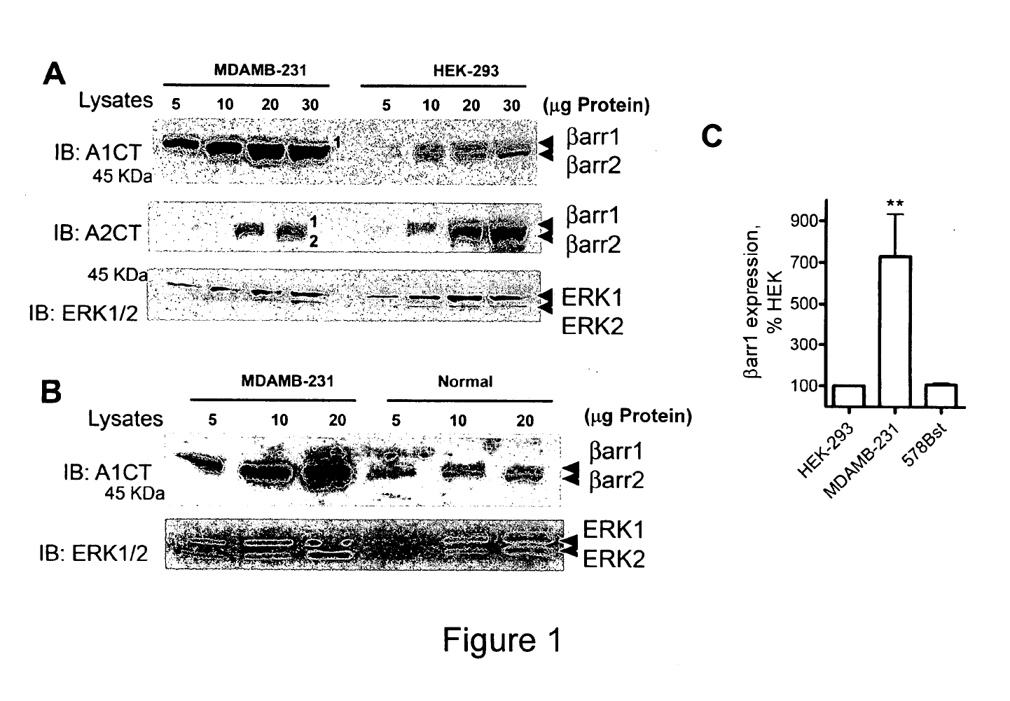

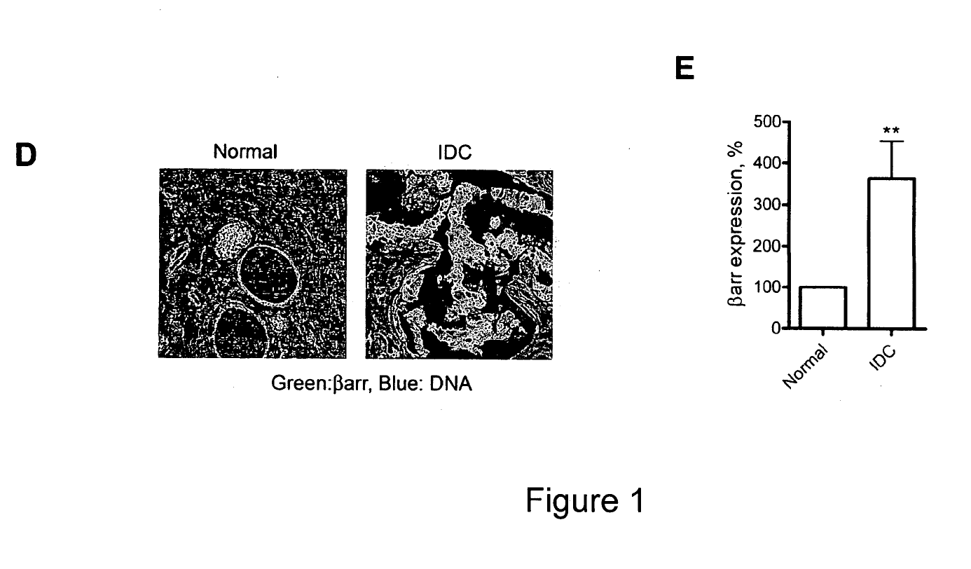

In the human genome, β-arrestin1 gene maps to chromosome locus 11q13, which is often amplified in breast cancer (Chuaqui et al, Am. J. Pathol. 150:297-303 (1997), Letessier et al, BMC Cancer, pg. 245 (2006), Rosa-Rosa et al, Breast Cancer Res. Treat. (2009)). While β-arrestin1 overexpression promotes tumor growth in mice (Zou et al, Faseb J. 22:355-364 (2008)), transcriptome and gene, profiling studies conducted thus far do not identify an increase in β-arrestin mRNA in breast cancer (Ma et al, Proc. Natl. Acad. Sci. USA 100:5974-5979 (2003), Niida et al, BMC Bioinformatics 10:71 (2009), Minn et al, Nature 436:518-524 (2005)). On the other hand, as shown in FIG. 1, a dramatic increase in β-arrestin1 protein levels was found in invasive breast carcinoma cells (MDAMB-231) when compared with non invasive cells (HEK-293) and normal breast epithelial cells (Hs 578Bst, ATCC). β-arrestin2 is also expressed in MDAMB-231 but at much lower levels than in either HEK-293 or Hs 578Bst. Additionally, in the noninvasive cells, β-arrestin2 is the more abundant isoform. The Western blot comparisons made between MDAMB-231, 578Bst and HEK-293 cells in FIG. 1 clearly indicates that O-arrestin1 is up-regulated only in the invasive carcinoma cells.

A determination was next made as to whether β-arrestin1 expression is increased in human cancer tissues. In general, breast cancer initiates as the premalignant stage of atypical ductal hyperplasia (ADH), progresses into the preinvasive stage of ductal carcinoma in situ (DCIS) and culminates in the potentially lethal stage of invasive ductal carcinoma (IDC) (Ma et al, Proc. Natl. Acad. Sci. USA 100:5974-5979 (2003)). Studies with laser capture microdissection (LCM) and DNA microarray have indicated that the pathologically discrete stages (ADH, DCIS and IDC) are highly similar to each other at the level of transcriptome (Ma et al, Proc. Natl. Acad. Sci. USA 100:5974-5979 (2003)). Because β-arrestin1 is a stable protein (half-life, 22 hours) and specific antibodies were available (Attramadal et al, J. Biol. Chem 267(25):17882-90 (1992)) β-arrestin1 protein levels were analyzed in normal and cancer tissue cores (MaxArray™ human breast carcinoma tissue microarray slides) by immunostaining with anti-β-arrestin1 (A1CT) antibody followed by Alexa Fluor® 488 secondary antibody and visualizing by high-resolution confocal microscopy (Zeiss LSM 510, and 40× or 100× oil immersion objective, FIG. 1D). Pixel intensity in each image for β-arrestin1 and DNA (DRAQ5™) channels were quantified using MetaMorph image analysis software. The amount of β-arrestin1 from each scan was normalized to the DNA levels (representing the total cellular content) for each section. About 70% of the IDC tissue sections analyzed had increased levels of β-arrestin1. As shown in FIG. 1E, β-arrestin1 expression was 3.5 to 4 fold higher in IDC than in normal breast specimens. Approximately two-three fold increase in β-arrestin expression was also seen in DCIS samples (n=3) when compared with normal tissues. Qualitatively identical immunostaining patterns were obtained with a second β-arrestin1 specific antibody (BD Biosciences) but very weak signals were observed with secondary antibody alone. Immunostaining with the anti-β-arrestin2 A2CT yielded much weaker signals than A1CT and hence the β-arrestin isoform detected in these sections is predominantly β-arrestin1.

### Knockdown of β-Arrestin Retards Colonization and Growth of Breast Carcinoma Cells in Experimental Metastasis Assays

Injection of fluorescence or bioluminescence tagged cancer cells into immune-compromised mice and monitoring the spread of tumor in the same animal for a considerable length of time is a recent advancement in the field of cancer biology due to the development of in vivo imaging techniques (Xenogen in vivo imaging systems). Indeed, a definitive method of assessing β-arrestin's role in vivo is to track the metastatic spread of MDAMB-231 cells lacking β-arrestins and compare the patterns with cells having normal β-arrestin expression. Accordingly, as described below, luciferase-tagged cancer cells with or without knockdown of β-arrestin were generated, assayed and the corresponding differences in the metastatic patterns analyzed.

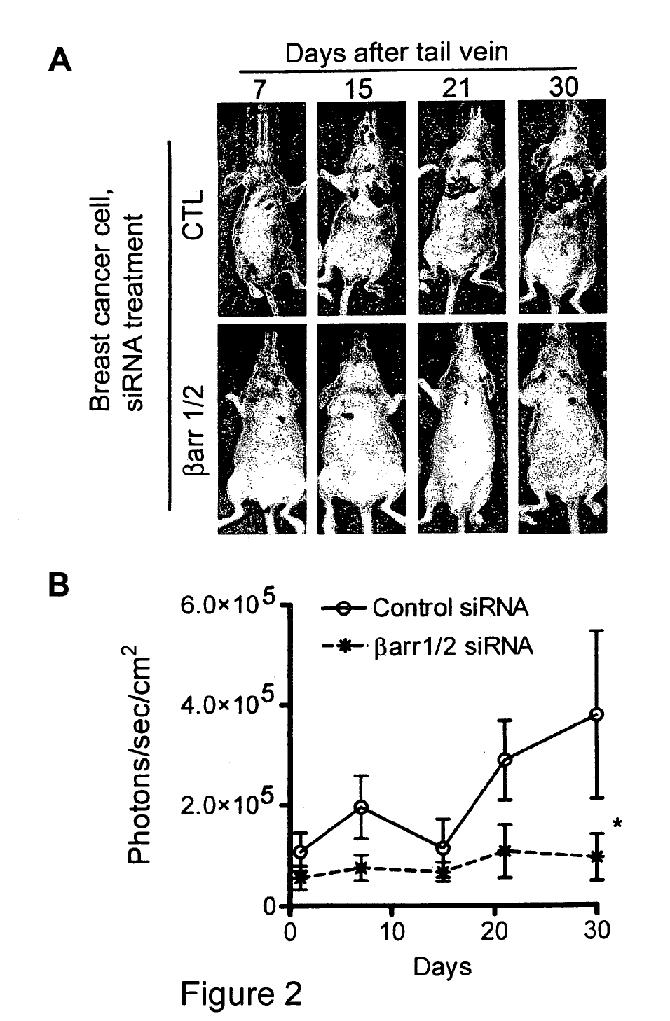

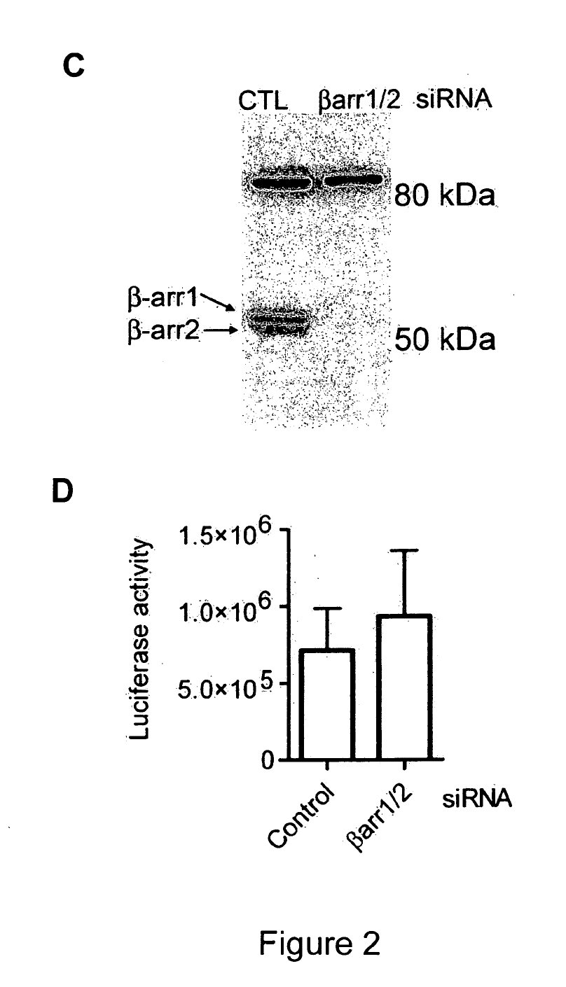

Transfection of MDAMB-231 cells with isoform-specific β-arrestin siRNA (Aim et al, Proc. Natl. Acad. Sci. USA 100:1740-1744 (2003), DeWire et al, Annu. Rev. Physiol. 69:483-510 (2007)) leads to a decrease in the levels of β-arrestin1 and β-arrestin2 by 75-85% and >90% respectively. In addition, simultaneous use of the two individual siRNAs is very effective in reducing the expression levels of both β-arrestins by 95-99%. With the achieved optimization, β-arrestin1 and 2 are consistently observed to remain down-regulated in MDAMB-231 cells up to two weeks or to three rounds of subcultivation, when both isoforms were downregulated. Knockdown of β-arrestins 1 and 2 individually did not result in such prolonged downregulation of protein levels. On the other hand, since both β-arrestins are indicated to play a role in cancer cell chemotaxis in vitro (Ge et al, J. Biol. Chem. 279:55419-55424 (2004), Fong et al, Proc. Natl. Acad. Sci. USA 99:7478-7483 (2002), Walker et al, J. Clin. Invest. 112:566-574 (2003), Hunton et al, Mol. Pharmacol. 67:1229-1236 (2005)) either individual or combined knockdown of the two isoforms could inhibit migration of cancer cells in vivo. Accordingly, 231-luc cells stably expressing luciferase were treated with either control siRNA or β-arrestin1/2 specific siRNA and tail vein injections into female nude mice were performed and bioluminescence imaging was carried out. Quite interestingly, stable lung colonization was observed only in control-treated mice but not in mice that received β-arrestin depleted cells (FIGS. 2A-2B) although the uptake of both control and β-arrestin1/2 treated cells in the lungs was nearly identical at 24 hours. After tail vein injections of these cells, the signals were detected and quantified over a period of 5 weeks using the Living Image acquisition and analysis software (Living Image®, Xenogen). The results are summarized in the graph presented in FIG. 2B for 9 control-mice (injected with control cells) and 10 β-arr-mice (injected with β-arrestin1/2 depleted cells). Five of the nine control-mice showed robust colonization at 30 days, and two displayed significant tumor growth, two had no signal, whereas four of the β-arr-mice showed negligible growth and six showed no growth of tumor cells. Statistical analysis using two-way ANOVA indicated a significant difference (p <0.05 between the control and β-arrestin1/2 at 30 days). Aliquots of cells used for the injection were immunoblotted for β-arrestin1/2 levels and analyzed for luciferase expression. As depicted in FIGS. 2C-2D, O-arrestins 1 and 2 were completely knocked down and β-arrestin1/2 knockdown cells had 15-20% more luciferase activity than control cells. These data suggest that β-arrestins play an important role in the survival and metastatic spread of breast cancer cells in vivo.

### β-Arrestin1 Expression is Critical for the Viability of Breast Carcinoma Cells Under Hypoxic Stress

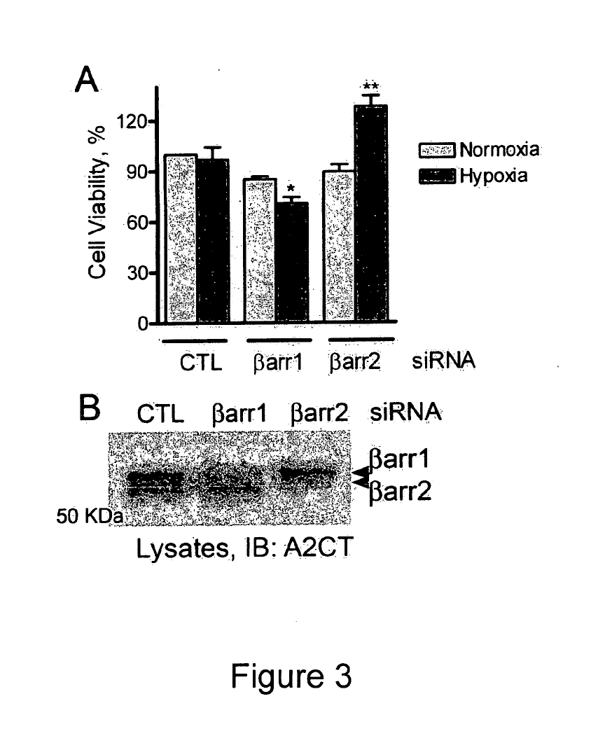

Although the above in vivo approach corroborates the overall importance of β-arrestins 1 and 2 in breast cancer metastasis, it does not address the individual roles of β-arrestins in viability and growth of cancer cells. To discern whether expression of individual β-arrestin isoform affects cancer cell viability, CellTiter-Glo® (Promega) luminescent cell viability assay was performed on breast carcinoma cells transfected with siRNA targeting no mRNA (control), β-arrestin1 or β-arrestin2. This assay is based on the quantification of the cellular ATP present, which indicates the presence of metabolically active cells. No significant differences were observed between control and β-arrestin knockdown conditions suggesting that cell viability was unaffected by depletion of β-arrestin1 or 2. Interestingly, when the cells were treated for 24 hours with 100 μM Cobalt Chloride (CoCl₂, a well-accepted hypoxia mimetic), cell viability was significantly reduced in β-arrestin1 depleted cells when compared with cells transfected with control siRNA (FIG. 3). In contrast, β-arrestin2 knockdown significantly increased cell viability during hypoxia (FIG. 3). Although hypoxia is toxic to normal as well as cancer cells, the latter undergo genetic and adaptive changes that allow survival and proliferation in a hypoxic environment. The data shown in FIG. 3 indicate that β-arrestin1 expression might facilitate cell survival during hypoxia and have a putative role in cell proliferation in a hypoxic environment by influencing adaptive gene programming via its signaling roles. These data also indicate a functional reciprocity of the two β-arrestin isoforms in regulating cell viability during hypoxia.

β-Arrestin1 Interacts with the Oxygen-Regulated Transcription Factor HIF-1α

The hypoxia-inducible factor-1 (HIF-1) is recognized as the master transcriptional switch during hypoxia, and activates >100 genes crucial for the adaptation to low oxygen tension (Semenza, Sci STKE cm8 (2007)). The HIF-1 transcription factor is a heterodimer consisting of the oxygen-regulated HIF-1α subunit and oxygen-insensitive HIF-1β subunit (aka aryl hydrocarbon receptor nuclear translocator, ARNT) (Wang et al, Proc. Natl. Acad. Sci. USA 92:5510-5514 (1995), Jiang et al, J. Biol. Chem. 271:17771-17778 (1996)). Under normoxia, HIF-1α is hydroxylated at specific proline residues, which leads to its ubiquitination by the E3 ubiquitin ligase and tumor suppressor pVHL (Maxwell et al, Nature 399:271-275 (1999)). Consequently, HIF-1α subunit is continuously degraded by the 26S proteasomal machinery. During hypoxia, prolyl hydroxylation does not occur and hence HIF-1α is not ubiquitinated and degraded. Stabilized HIF-1α translocates to the nucleus, heterodimerizes with HIF-1β to form a functional transcription factor and binds to specific promoter regions known as hypoxia responsive elements (HRE) to induce transcription of many genes especially those required for angiogenesis (e.g., VEGF), cell survival (e.g. insulin-like growth factor, IGF2), glucose metabolism (e.g. glucose transporter, GLUT1) and invasion (e.g. transforming growth factor α, TGFα) (Semenza, Sci STKE cm8 (2007)). It is also suggested that optimal HIF-1 activity requires p300 binding (Arany et al, Proc. Natl. Acad. Sci. USA 93:12969-12973 (1996), Kallio et al, Embo J. 17:6573-6586 (1998), Ebert and Bunn, Mol. Cell Biol. 18:4089-4096 (1998)) and might involve other juxtaposed transcriptional elements such as β-1 (Kvietikova et al, Nucleic Acids Res. 23:4542-4550 (1995), Ke and Costa, Mol. Pharmacol. 70:1469-1480 (2006)). Based on the effect α-arrestin1 knockdown on cell survival (FIG. 3) and on the knowledge about its nuclear localization and nuclear function in forming a complex with p300 Kang et al, Cell 123:833-847 (2005)), it was hypothesized that β-arrestin1 could be modulating the transcriptional activity of HIF-1α, thus, regulating the growth and survival of breast carcinoma cells

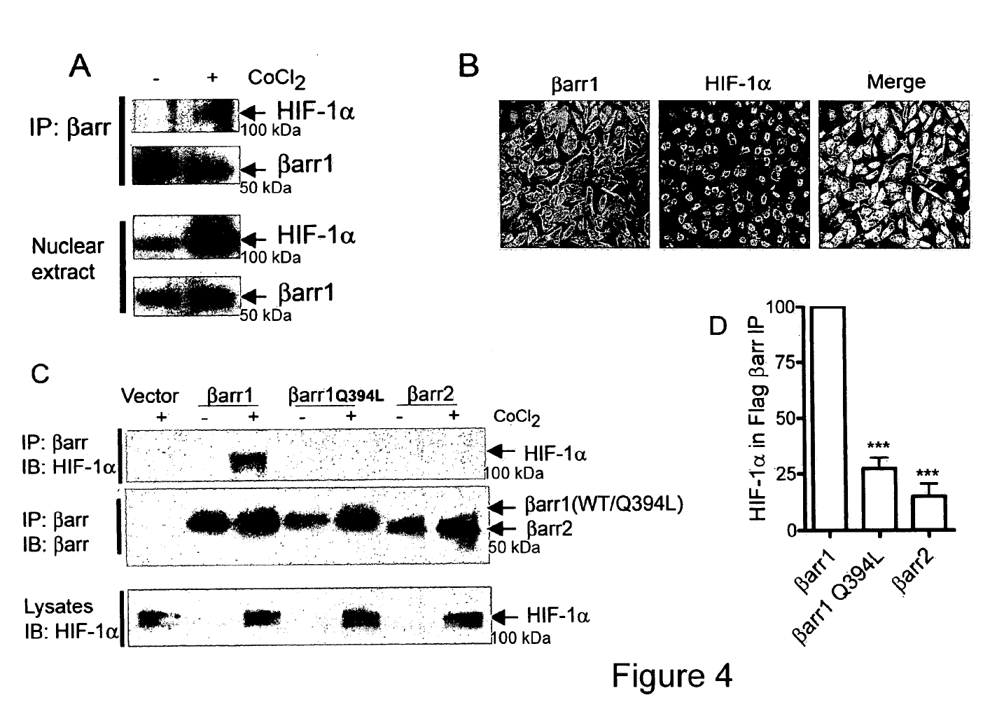

As a first step to test the above hypothesis, an interaction between β-arrestin1 and HIF-1α during hypoxia was investigated. To assess whether β-arrestin1-HIF1α interaction occurs in cells expressing endogenous amounts of the two proteins, nuclear extracts were prepared from breast carcinoma cells treated with CoCl₂ and the interaction tested by immunoprecipitation and immunoblotting (FIG. 4A). HIF-1α was detected in β-arrestin immunoprecipitates (IPs) from CoCl₂ treated cells, but neither in untreated samples nor in IPs with control IgG, indicating that there is a specific interaction between β-arrestin1 and stabilized HIF-1α. Colocalization of β-arrestin1 and HIF-1α was detected in the nucleus by immunostaining the two proteins with specific antibodies followed by confocal microscopy (FIG. 4B). The exclusive cytoplasmic distribution of β-arrestin2 is attributed to the presence of a nuclear export signal (NES) that is absent in β-arrestin1 (Scott et al, J. Biol. Chem. 277:37693-37701 (2002), Wang et al, J. Biol. Chem. 278:11648-11653 (2003)). Introduction of this NES in β-arrestin 1 (βarr1Q394L) changes its subcellular distribution to be totally cytoplasmic (Scott et al, J. Biol. Chem. 277:37693-37701 (2002), Wang et al, J. Biol. Chem. 278:11648-11653 (2003)). When β-arrestin1, β-arrestin1Q394L and β-arrestin2 were compared for their binding to HIF-1α, only the wild type β-arrestin1 formed a robust complex with HIF-1α (FIGS. 4C-4D) thus indicating that β-arrestin1-HIF-1α complexes are either formed or stabilized predominantly in the nuclear compartment.

### B-Arrestin1 Regulates the Transcriptional Activity of HIF-1α

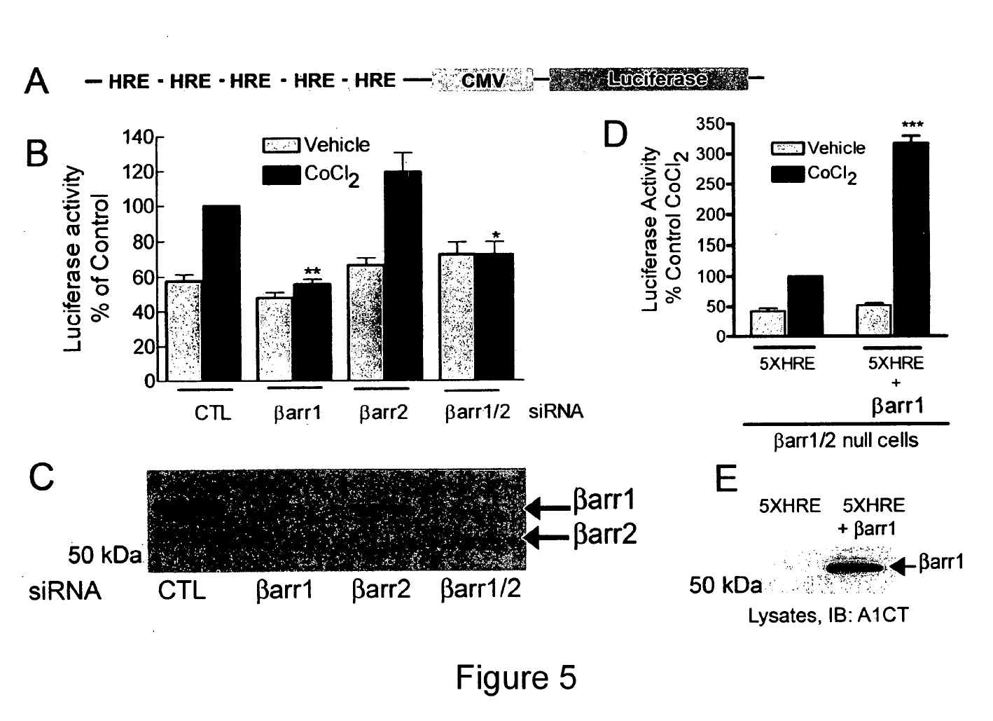

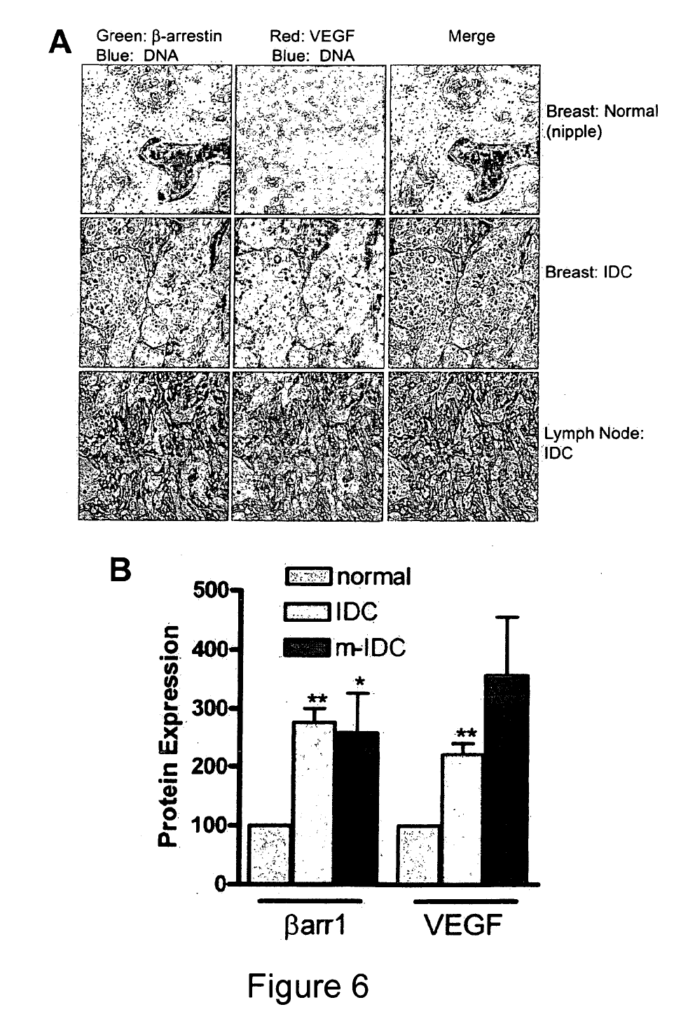

To test if the above β-arrestin1-HIF-1α interaction has functional consequences, an analysis was made of the effect of β-arrestin1 expression on HIF-1-mediated transcription during hypoxia. One of the most characterized HIF-regulated genes is the potent endothelial mitogen, VEGF-A, which regulates endothelial cell proliferation and blood vessel formation in both normal and cancerous tissues (Liu et al, Circ. Res. 77:638-643 (1995)). The VEGF-A gene contains a HRE in its 5′ UTR (untranslated region) and hypoxia induces a rapid and sustained increase in VEGF-A mRNA levels. To assess if HIF-1 dependent VEGF induction involves β-arrestin, a reporter based assay was used as follows. Breast carcinoma cells (MDAMB-231) were transfected with a plasmid encoding five copies of hypoxia-responsive elements (5×HRE) derived from the 5′ UTR of the human VEGF gene fused in frame to firefly luciferase gene (5×HRE/FL/pCDNA3) (FIG. 5A). The same cells were also transfected with pRL-tk-luc (that encodes Renilla luciferase under the control of the thymidine kinase promoter) along with siRNA targeting no known mRNA (control), β-arrestin1, β-arrestin2 or β-arrestin1/2. Sixty hours post transfection, cells were treated with 300 μM CoCl₂ for 5 hours. At the end of the incubation, cell lysates were prepared and assayed sequentially for the Firefly and Renilla luciferase activities. 5×HRE reporter transfection alone leads to basal activity in MDAMB-231 cells. Nonetheless, treatment of cells with CoCl₂ significantly increased the response (FIG. 5B). This CoCl₂-induced increase was not observed in cells with β-arrestin1 or β-arrestin1/2 knockdown (FIGS. 5B-5C). Not surprisingly, β-arrestin2 depletion did not decrease but slightly increased the reporter activity. These data suggest that HIF-1 dependent transcriptional activity during hypoxia is regulated specifically by β-arrestin1 expression in MDAMB-231 cells. Additionally, HIF-1α mediated transcriptional activity was tested under β-arrestin1 null and replete conditions in a β-arrestin1/2 double knockout cell line (Kohout et al, Proc. Natl. Acad. Sci. USA 98:1601-1606 (2001)). As shown in FIG. 5D, although a 1.8 fold CoCl₂-induced increase in HIF-1 transcriptional response was found in these β-arrestin null fibroblasts, restoration of β-arrestin1 expression did lead to a dramatic increase (6-7 fold) in HIF-1 mediated transcription during hypoxia. Thus, β-arrestin1 does augment HIF-1 directed transcription and there could be potential β-arrestin1-dependent signals with vital roles during hypoxia.

Increase in VEGF Levels in Invasive Ductal Carcinoma Correlates with Increased β-Arrestin1 Levels

Because an increase in β-arrestin1 expression was observed in IDCs (FIG. 1), and because β-arrestin1 expression correlated with HIF-1 transcriptional activity in MDAMB-231 cells (FIG. 5), the question presented was whether this would be reflected in the levels of the downstream target of HIF-1, VEGF, in IDCs. An analysis was made of β-arrestin1 and VEGF protein expression by immunostaining with the antibodies anti-β-arrestin1 (A1CT) and anti-VEGF-A (mouse monoclonal C-1), followed by respective secondary antibodies, one conjugated to Alexa Flour® 488 and the other to Alexa Flour® 594. Confocal images using LSM 510 microscope and a 40× oil objective. All the tissue sections within an experiment were scanned with the same instrumental setting for image acquisition and each experiment included tissue sections that were stained only with secondary antibodies that constituted the negative control. The first scans were obtained for sections of normal breast and following this images were acquired in a random order for different samples in a tissue microarray that contained 50 tissue cores representing a collection of twenty-four IDCs, ten metastatic-IDCs from lymph node, three lobular carcinomas, two medullary carcinomas, one papillary carcinoma and ten normal non-neoplastic tissues from breast cancer patients. Tissue arrays from two different sources were analyzed: IMGENEX HISTO-Array™ and Zymed's MaxArray™ with a total of 50 cores in each. Collected images were quantified for pixel intensity for each channel using the MetaMorph software. A representative set of such confocal images for normal, IDC and metastatic IDC is shown in FIG. 5A. The data acquired for β-arrestin1 and VEGF immunostaining for normal breast (n=10 tissue samples, 22 images), IDC (n=40 tissue samples, 80 images) and metastatic carcinoma in lymph node (n=10 tissue samples, 12 images) is summarized as bar graphs in FIG. 5B. Although VEGF expression varied from none to very high levels among the different cancer samples, Overall both β-arrestin1 and VEGF levels were increased more than three fold and significantly higher (p<0.01) in IDC samples than in normal breast tissues. In contrast, there was no direct relationship between the increase in β-arrestin1 and presence of estrogen receptor, progesterone receptor or expression of p53. These findings suggest that an increase in β-arrestin expression could enhance HIF-1 dependent transcription of VEGF-A in neoplastic and metastatic breast cancer.

Inhibition of VEGF Secretion by Thalidomide Results from a Disruption of β-Arrestin1-HIF-1 Signaling

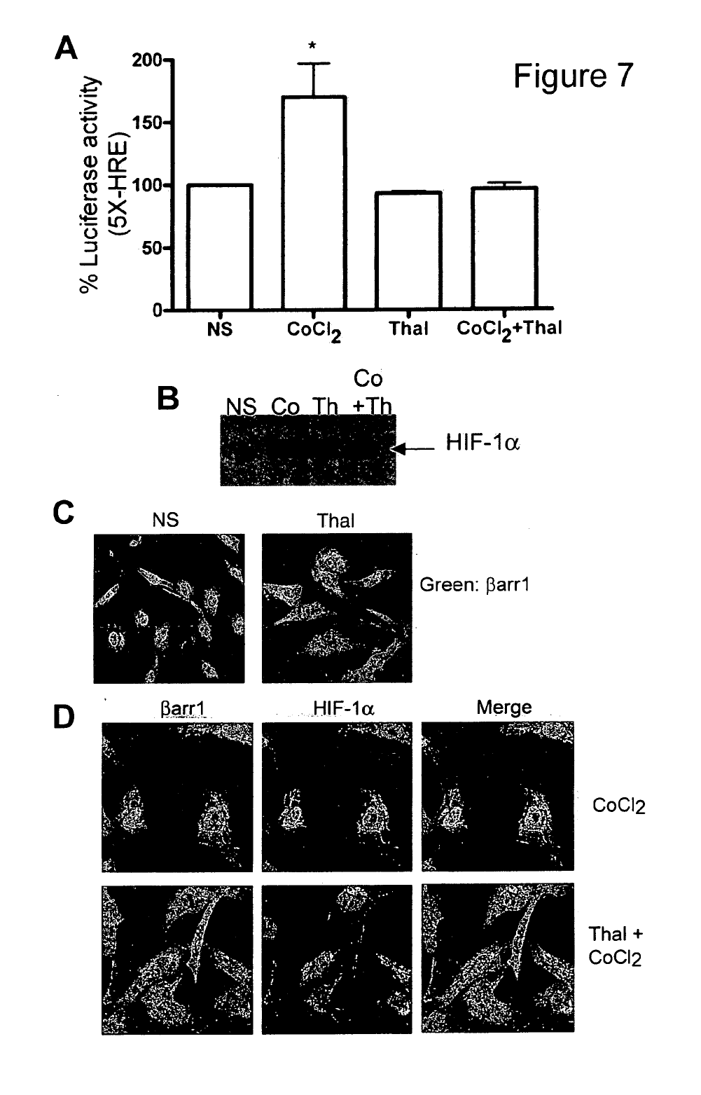

The immunomodulatory drug thalidomide was previously shown to suppress angiogenesis, although the mechanism was not clearly laid out (D'Amato et al, Proc. Natl. Acad. Sci. USA 91:4082-4085 (1994), Holaday and Berkowitz, Mol. Interv. 9:157-166 (2009), Figg, Clin. Pharmacol. Ther. 79:1-8 (2006)). It was further suggested that thalidomide inhibits secretion of VEGF from tumors and bone marrow stromal cells leading to decreased endothelial cell migration and adhesion ((Dredge et al, Br. J. Cancer 87:1166-1172 (2002), Vacca et al, J. Clin. Oncol. 23:5334-5346 (2005)). When MDAMB-231 cells were treated with CoCl₂ along with thalidomide (10 μM), a complete inhibition of HIF-1 dependent transcriptional response was observed as measured by 5×HRE luciferase reporter activity (FIG. 7A). Paradoxically, HIF-1α stabilization during hypoxia appeared to be normal (FIG. 7B). However, when β-arrestin1 distribution in thalidomide treated cells was analyzed, a predominant cytoplasmic translocation of β-arrestin1 from the nucleus was observed and only 10-15% protein remained in the nucleus as assessed by immunostaining (FIG. 7C). Furthermore, while treatment of CoCl₂ alone induced a robust HIF-1α and β-arrestin1 colocalization in the nuclear compartment (upper panels of FIG. 7D), addition of thalidomide and CoCl₂ resulted in relocation of β-arrestin1-HIF-1 complexes to the perinuclear compartments. Under these conditions, the effect of nuclear exclusion was more prominent in the case of fi-arrestin1 than the stabilized HIF-1α. These data strongly suggest that β-arrestin1 is a crucial regulator of HIF-1 dependent transcription and VEGF secretion and that drugs that can induce its translocation to the cytoplasm could prove useful in reducing gene transcription during hypoxia and serve as inhibitors of angiogenesis and, therefore, useful in the treatment of breast cancer.

What is claimed is:

1. A method of treating breast cancer comprising administering to a patient in need thereof an amount of an agent that modulates signal transduction regulated by β-arrestin sufficient to effect said treatment.

2. The method according to claim 1 wherein said agent modulates signal transduction regulated by β-arrestin1.

3. The method according to claim 1 wherein said agent binds β-arrestin and modifies the interaction between β-arrestin and its signaling partner.

4. The method according to claim 3 wherein said agent is an antibody, or antigen binding fragment thereof.

5. The method according to claim 3 wherein said agent is an aptamer.

6. The method according to claim 1 wherein said agent inhibits expression of β-arrestin.

7. The method according to claim 6 wherein said agent is an siRNA or an miRNA.

8. The method according to claim 1 wherein said patient is a human.

9. The method according to claim 8 wherein said breast cancer is a breast carcinoma.

10. The method according to claim 9 wherein said carcinoma is invasive ductal carcinoma.

11. The method according to claim 1 wherein said agent inhibits β-arrestin1-HIF-1 signaling.

12. Agent that modulates signal transduction regulated by β-arrestin for use in the treatment of breast cancer in a patient by administering to said patient an amount of said agent sufficient to effect said treatment.

13. Use of a therapeutically effective amount of an agent that modulates signal transduction regulated by β-arrestin for the manufacture of a medicament for the treatment of breast cancer in a patient.
